(19) United States
(12) Patent Application Publication
(10) Pub. No.: US 2026/0108670 A1
(43) Pub. Date: Apr. 23, 2026

(54) ASEPTIC MANUFACTURING SYSTEM AND METHOD FOR DOSING AND PACKAGING OF A PHARMACEUTICAL PRODUCT

(71) Applicant: Julien Daniel Marechal, Houston, TX (US)

(72) Inventor: Julien Daniel Marechal, Houston, TX (US)

(21) Appl. No.: 18/919,279

(22) Filed: Oct. 17, 2024

(51) Int. Cl. A61M 5/00; A61B 50/33

(52) U.S. Cl. CPC A61M 5/002; A61B 50/33

(57) ABSTRACT

An aseptic system includes a flexible enclosure and a tray assembly configured to be disposed within the flexible enclosure. The tray assembly includes a first tray configured to support a plurality of container bodies, where each of the plurality of container bodies has an opening into a chamber. The tray assembly also includes a second tray configured to support a plurality of container seals, where each of the plurality of container seals is configured to seal the opening of a respective one of the plurality of container bodies. The aseptic system also includes a dosing system including a dosing conduit extending through the flexible enclosure and the second tray, where the flexible enclosure is configured to enable movement of the second tray with the dosing conduit to dispense a pharmaceutical product into the chamber of each of the plurality of container bodies.

### BACKGROUND

The present disclosure relates generally to an aseptic manufacturing system and method for dosing and packaging of a pharmaceutical product.

This section is intended to introduce the reader to various aspects of art that may be related to various aspects of the present disclosure, which are described and/or claimed below. This discussion is believed to help provide the reader with background information to facilitate a better understanding of the various aspects of the present disclosure. Accordingly, it is understood that these statements are to be read in this light, and not as admissions of prior art.

The aseptic manufacturing of pharmaceutical products (e.g., dosing and packaging of medication) prevents contamination by excluding microorganisms during production. To ensure the exclusion of microorganisms, the manufacturing and packaging processes are performed using sterilized materials in a controlled, sterilized environment designed to prevent microbial contamination of the pharmaceutical product. In some cases, such sterilization may require repetition of certain steps and/or specialized equipment (e.g., an isolator or laminar air flow) that may be bulky and expensive and may require continuous monitoring and maintenance to ensure the sterility of the environment. As such, it may be recognized that an improved aseptic manufacturing system for dosing and packaging of medication is desired.

### BRIEF DESCRIPTION

Certain embodiments commensurate in scope with the originally claimed subject matter are summarized below. These embodiments are not intended to limit the scope of the disclosure, but rather these embodiments are intended only to provide a brief summary of certain disclosed embodiments. Indeed, the present disclosure may encompass a variety of forms that may be similar to or different from embodiments set forth below.

In an embodiment, an aseptic system includes a flexible enclosure and a tray assembly configured to be disposed within the flexible enclosure. The tray assembly includes a first tray configured to support a plurality of container bodies, where each of the plurality of container bodies has an opening into a chamber. The tray assembly also includes a second tray configured to support a plurality of container seals, where each of the plurality of container seals is configured to seal the opening of a respective one of the plurality of container bodies. The aseptic system also includes a dosing system including a dosing conduit extending through the flexible enclosure and the second tray, where the flexible enclosure is configured to enable movement of the second tray with the dosing conduit to dispense a pharmaceutical product into the chamber of each of the plurality of container bodies.

In an embodiment, a method includes supporting a plurality of container bodies in a first tray of a tray assembly, where each of the plurality of container bodies has an opening into a chamber. The method also includes supporting a plurality of container seals in a second tray of the tray assembly, where each of the plurality of container seals is configured to seal the opening of a respective one of the plurality of container bodies. The method also includes positioning the tray assembly within a flexible enclosure. The method also includes extending a dosing conduit of a dosing system through the flexible enclosure and the second tray, where the flexible enclosure is configured to enable movement of the second tray with the dosing conduit to dispense a pharmaceutical product into the chamber of each of the plurality of container bodies.

In an embodiment, an aseptic system includes at least one component of a tray assembly configured to be disposed within a flexible enclosure and a dosing system. The dosing system including a dosing conduit configured to extend through the flexible enclosure and the at least one component, where the flexible enclosure is configured to enable movement of the at least one component with the dosing conduit to dispense a pharmaceutical product into a chamber of each of a plurality of container bodies disposed in the tray assembly.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a diagram of a side view of an aseptic system for use in manufacturing of sterile injectables, according to an embodiment of the present disclosure;

FIG. 2 is a perspective diagram of an exploded top view of the aseptic system of FIG. 1 pivoted to a side position, according to an embodiment of the present disclosure;

FIG. 3 is a perspective diagram of an exploded side view of the aseptic system of FIG. 1 having a flexible enclosure, according to an embodiment of the present disclosure;

FIG. 4 is a diagram of a side view of the aseptic system of FIG. 1 during a dosing stage of manufacturing, according to an embodiment of the present disclosure;

FIG. 5 is a diagram of the aseptic system of FIG. 1 during a sealing stage of manufacturing, according to an embodiment of the present disclosure;

FIG. 6 is a diagram of the aseptic system of FIG. 1 for use in automated manufacturing of sterile injectables, according to an embodiment of the present disclosure; and

FIG. 7 is a diagram of the aseptic system of FIG. 1 for use in automated manufacturing of sterile injectables, according to an embodiment of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

As used herein, the term “sterile” may refer to an object, equipment, environment, medication, packaging, or other item being completely clean (i.e., free from dirt, debris, bacteria, or other contaminant or living microorganism). As used herein, the term “aseptic” may refer to an object, equipment, environment, medication, packaging, or other item being free from harmful contaminants such as harmful bacteria, viruses, or other harmful microorganisms. Further, the term “sterile” may be used herein to broadly encompass complete contamination eradication (e.g. the complete absence of viable microorganisms via the killing of all microbes with the potential to reproduce), whereas the term “aseptic” may be used herein to describe contamination prevention by ensuring the absence or the exclusion of microbes during processing or manufacturing.

As used herein, the term “sterile injectables” refers to medication or other pharmaceutical product that may be administered directly into the bloodstream or the tissue of a person (e.g. a patient). As used herein, the term “non-sterile drugs” may refer to drugs, medications, or other pharmaceutical product that is manufactured and/or administered in the form of a pill, a gel capsule, an aerosol, or the like.

As used herein, the term “ready-to-use” may refer to pre-sterilized materials, such as packaging or dosing materials, that may be used for the aseptic manufacturing of a pharmaceutical product. Such read-to-use materials do not need to be prepared prior to use for manufacturing and may, therefore, eliminate the need to maintain washing and sterilization equipment.

The present disclosure relates generally to aseptic manufacturing of pharmaceutical products, such as medication, in the form of, for example, sterile injectables. The aseptic manufacturing of sterile injectables requires using sterile materials in a sterile environment. To ensure such sterility, some manufacturers may perform laborious sterilization steps and/or use bulky sterile enclosures, which may be expensive and time consuming and may need continuous monitoring and maintenance.

The presently disclosed systems and methods include a sterilized, ready-to-use aseptic manufacturing system for dosing and packaging of a pharmaceutical product. The system may have a tray assembly with multiple trays to house one or more pharmaceutical containers (e.g., vials, bottles, bags, cartridges, ampules, syringes, etc.) and one or more seals or caps for the containers. A dosing needle configured to inject the pharmaceutical product into the container may be inserted in the tray housing the seals and may be connected to a conduit or hosing for transporting the product from a source into the container. The system may be contained in a flexible enclosure (e.g., flexible bag) that enables the movement of the dosing needle and the tray housing the seals to dispense the product into the containers. Once filled, the containers may be sealed by, for example, pressing the tray housing the seals down on the tray housing the containers (e.g. press fitted). Once the filled containers are sealed, the flexible enclosure may be removed and disposed along with the one or more trays and/or the dosing needle and conduit.

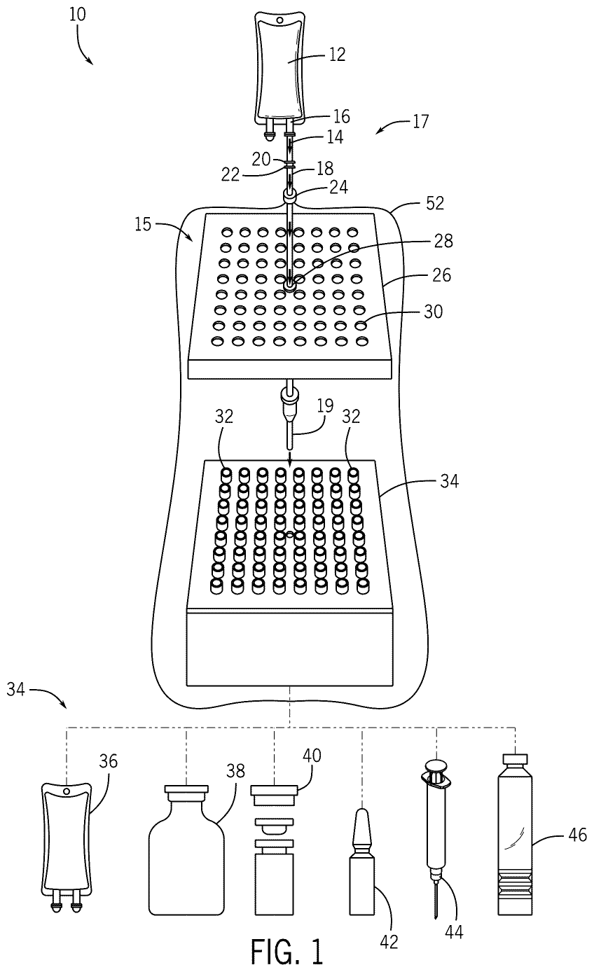

By way of introduction, FIG. 1 is a diagram of a side view of an aseptic system 10 for the manufacturing of sterile injectables. The system 10 may include a fluid supply 12, which may serve as the source of a fluid pharmaceutical product 14, such as a medication manufactured for medical treatment of a patient. For example, the fluid 14 may be in the form of a liquid, an elixir, a solution, a suspension, a syrup, or other flowing substance that is able to flow through a conduit and/or be dispensed via a needle. The fluid supply 12 may include one or more fluid tanks, pumps, valves, filters, or any combination thereof.

The fluid supply 12 may include or be coupled to one or more fluid supply conduits, tubing, or other type of channel 16 through which the fluid 14 may pass from the fluid supply 12 to a dosing system 17. The dosing system 17 may include or be coupled to one or more dosing conduits, tubing, or channels 18 and/or a dosing needle 19 configured to dispense the fluid 14 in a certain quantity or amount. To maintain sterility between the fluid supply 12 and the dosing system 17, the fluid supply 12 and the dosing system 17 may be fluidly coupled via one or more sterile and/or aseptic fasteners, connectors, or ports (e.g., complimentary connectors 20, 22) that allow the fluid 14 to flow from the fluid supply conduit 16 to the dosing conduit 18 with minimal risk of contamination. In one embodiment, the dosing conduit 18 and the fluid supply conduit 16 may be fluidly coupled via one or more sterile connectors 20, 22. For example, the sterile connectors 20, 22 may include complimentary male and female parts or mating halves that “click” or fasten directly together to allow sterile fluid transfer from the fluid supply 12 to the dosing system 17. Each connector 20, 22 may include one or more protective layers, such as a film, cover, or membrane, that may be removed after fastening. In this way, sterility may be maintained during the connection process to ensure no contamination of the fluid 14. In some embodiments, the one or more sterile connectors 20, 22 may connect to one or more additional conduits or tubing rather than fastening directly together.

The system 10 may also include an enclosure 52, which may be at least partially or entirely flexible to enable movement of the dosing system 17. The dosing system conduit 18 may be inserted into the enclosure 52 and sealed at the insertion point via one or more seals 24 (e.g., annular seals). As such, the enclosure 52 may contain at least a portion of the dosing conduit 18. The enclosure may also contain a tray assembly 15 having at least two trays. At least one tray of the tray assembly 15 may be a seal tray 26 (e.g. a press-fit cap nest), which may include a plurality of container seals 30 (e.g., closures, caps, lids, stoppers, or other type of container “tops”) configured to seal the opening of one or more containers after filling (e.g., dosing) a chamber of a container body. In some embodiments, the dosing conduit 18 may extend through the seal tray 26 (e.g., in one side and out the other side). In such embodiments, the dosing conduit 18 may be fixed in place by one or more seals, retainers, and/or stoppers 28 that block inadvertent movement of the dosing conduit 18 during operation of the dosing system 17.

The dosing conduit 18 may be additionally coupled to a dosing needle 19 configured to dispense the fluid 14 into one or more containers or container bodies 32 (e.g. vials, bags, cartridges, ampules, syringes, bottles, etc.). The dosing system 17 may include a peristaltic, volumetric, or time pressure pump configured to dispense the fluid 14 at a certain quantity or amount (i.e., dosage). The one or more container bodies 32 may be disposed in a container tray 34 of the tray assembly 15 (e.g., a container nest). The one or more container bodies 32 may be packaging used for the manufacturing of sterile injectables (e.g., medications that are administered directly into the bloodstream or tissues of the body). For example, the one or more container bodies 32 in the container tray 34 (e.g., the container nest) may be in the form of a bag 36, a bottle 38, a vial 40, an ampule 42, a syringe 44, a cartridge 46, or any combination thereof.

The system 10 (e.g., the tray assembly 15, the dosing system 17, and the enclosure 52) may be sterilized prior to use. For example, each part or component of the system (e.g., the one or more trays of the tray assembly 15, the fluid supply conduit 16, the dosing conduit 18, the one or more seals 30, the one or more container bodies 32, the dosing needle 19, etc.) may be sterilized individually prior to incorporation into the system 10. In some embodiments, the system 10 may be sterilized as a whole after each component is incorporated into the system 10 prior to use of the system 10. Sterilization may be performed to protect the fluid 14 and the packing (e.g., the container bodies 32 and the seals 30) from contamination during manufacturing. Using sterile equipment maintains the aseptic environment needed during manufacturing. In this way, the system 10 may be implemented as a ready-to-use pharmaceutical manufacturing system. For example, in an embodiment, the system 10 may be implemented as an aseptic fill-finish system in which sterility is maintained across all materials, packaging, process steps, and outputs throughout the manufacturing process, which may be advantageous for the manufacturing of pharmaceutical products that cannot tolerate terminal sterilization (e.g., final-step sterilization via steam and/or radiation) where the product in the container may be sterilized together before shipping. As such, all parts of the system 10 (e.g., the fluid supply 12, the fluid 14, the connectors and seals 20, 22, 24, 28, the conduits, 16, 18, the trays 26, 34, the containers and seals 30, 32, the dosing needle 19, and the enclosure 52) may be sterilized either individually or after assembly to ensure the system 10 is free of contaminants. The components of the system 10 may be sterilized by one or more sterilization systems or methods, including steam, radiation, ethylene oxide gas, hydrogen peroxide gas plasma, filtration, liquid chemicals (e.g., peracetic acid), ozone, ultraviolet (UV) light, or other method of eliminating all living organism from a material.

The system 10 may maintain an aseptic environment throughout manufacturing by conducting the dosing process within the enclosure 52. For example, the container bodies 32 may be disposed in the container tray 34, such that an opening of the container body 32 is exposed to the dosing needle 19 for dosing (e.g., filling a chamber of the container body 32). In some embodiments, the dosing of each container body 32 may be achieved by moving the dosing needle 19 (e.g. manually or automatically) to position it over the opening of the container body 32. For example, the enclosure 52 may comprise or consist of a type of material that is flexible (e.g., plastic), such that the seal tray 26 may be freely moved inside the enclosure 52. For example, the enclosure 52 may comprise or consist of a plastic bag. In certain embodiments, the enclosure 52 has a wall that is defined by a flexible wall portion, a rigid wall portion, or a combination thereof. For example, the flexible wall portion may be at least 20, 30, 40, 50, 60, 70, 80, 90, 95, or 100 percent of the wall of the enclosure 52. In some embodiments, the enclosure 52 may include one or more rigid wall portions separate from and/or integrated with tray walls of the trays 26 and/or 34. In some embodiments, the enclosure 52 may be completely separate from the trays 26 and 34, except for the connection with the dosing system 17. In certain embodiments, the enclosure 52 may have the flexible wall portion at least between the trays 26 and 34, such that the flexible wall portion enables movement of the dosing system 17 and the tray 26 relative to the tray 34.

In this way, the enclosure 52 (e.g., at least partially or entirely flexible) may enable the movement of the seal tray 26 with the dosing conduit 18 and the dosing needle 19 to dispense the fluid 14 into the chamber of each container body 32. In particular, the enclosure 52 enables a user or a machine (e.g., robotic arm assembly) to manually or automatically move the seal tray 26, relative to the container tray 34, with the dosing conduit 18 from one container body 32 to another for dosing the fluid 14 into the respective container body 32 until all container bodies 32 are filled with the fluid 14. Additionally or alternatively, the enclosure 52 may enable the movement of the container tray 34 relative to the dosing system 17 and the seal tray 26, so as to position each container body 32 concentrically with the dosing needle 19 to dispense the fluid 14 into the chamber of each container body 32.

The enclosure 52 may consist of a type of material that is transparent and/or translucent, so as to allow an operator to see into the enclosure 52 while dosing. In some embodiments, the enclosure 52 may be opaque and/or colored (e.g., yellow, red, blue or any combination thereof). In some embodiments, one portion of the enclosure 52 may be transparent and/or translucent while another portion is opaque. In certain embodiments, the enclosure 52 may have a thickness between 10 μm up to 1 millimeter (e.g. less than 1 mil up to 40 mils). In another embodiment, the enclosure 52 may be approximately 1, 2, 3, 4, or 5, 6, 7, 8, 9, or 10 mils thick plus/minus 1, 2, or 3 mils at least in the flexible wall portion of the enclosure 52. For example, the enclosure 52 may be approximately 1 to 10 mils, 1 to 5 mils, or 5 to 10 mils thick. Additionally, in certain embodiments, the enclosure 52 may be made of medical grade materials, such as polyethylene (e.g., Low Density Polyethylene (LDPE) or High Density Polyethylene (HDPE)), polypropylene, nylon, various polymers, ethylene vinyl acetate (EVA), fluoropolymer, or any combination thereof.

Once dosing is complete, the seal tray 26 may be aligned with the container tray 34 manually by a user and/or assisted by a machine (e.g., robotic arm assembly), such that each seal 30 aligns with each opening of each respective container body 32. Once the seal tray 26 and the container tray 34 are aligned, each container body 32 may be sealed or closed by fastening, or otherwise attaching, the seals 30 (e.g., the caps, stoppers, lids, “tops”) to the opening of the container bodies 32. This may be achieved, for example, manually by a user and/or assisted by a machine (e.g., robotic arm assembly) by lowering the seal tray 26 towards the container tray 34, aligning each seal 30 with the opening of each container body 32, and applying pressure to the seal tray 26 to cause the seals 30 to seal the container bodies 32. In this way, each tray of the tray assembly 15 may be coupled together in an axial direction (e.g., on a vertical axis) relative to axes of the container bodies 32, such that the seals 30 are configured to seal the openings of the container bodies 32 in response to coupling together the seal tray 26 and the container tray 34 in the axial direction. After the container bodies 32 have been sealed, there may not be a risk of contamination. As such, the enclosure 52 may be removed and/or disposed and the container bodies 32 (e.g., vials) may be removed for labeling, inspection, and/or shipping. Certain aspects of the above-described aseptic system 10 are described in more detail below.

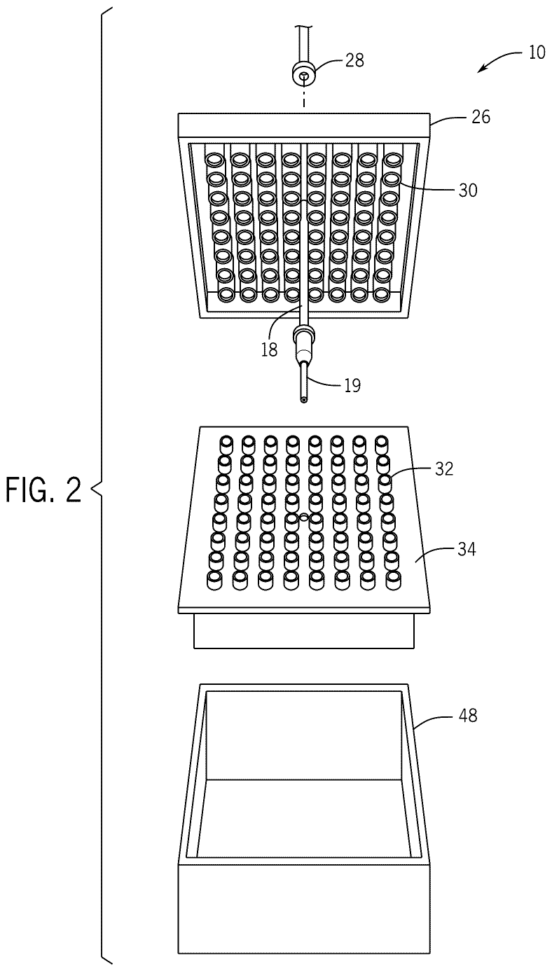

FIG. 2 is a exploded perspective top view of the aseptic system 10 of FIG. 1 pivoted to a side position. As demonstrated in FIG. 2, the dosing conduit 18 coupled to the dosing needle 19 may extend through the seal tray 26 housing the one or more seals 30 (e.g., caps, lids, tops, stoppers, etc.). The positioning of the dosing conduit 18 may be fixed in place by one or more stoppers 28 that block inadvertent movement of the dosing conduit 18 during operation. For example, an extension distance of the dosing conduit 18 through the seal tray 26 may be fixed in place by the stopper 28, such that the dosing conduit 18 does not slip or budge such that the dosing conduit 18 and the seal tray 26 move co-dependently (e.g., in a same field of direction) rather than independently. In some embodiments, an extension distance of the dosing conduit 18 through the seal tray 26 may be adjusted by, for example, manually loosening and tightening the stopper 28. For example, the stopper 28 may include a threaded nut coupled to a threaded sleeve, wherein a compression washer and/or seal is disposed between the threaded nut and the threaded sleeve. It should be noted that FIG. 2 illustrates the seal tray 26 with the dosing conduit 18 and dosing needle 19 inserted and extending through the seal tray 26 facing up, such that the seals 30 are facing away from the opening of the container bodies 32. However, the orientation shown in FIG. 2 is for demonstration purposes to illustrate how the dosing conduit 18 may extend through the seal tray 26. During operation, in some embodiments, the seal tray 26 with the dosing conduit 18 and the dosing needle 19 may be facing down (e.g., towards the container bodies 32), such that the seals 30 are facing the opening of the container bodies 32.

In some embodiments, one or more of the trays in the tray assembly 15 may have a nested format such that the contents of one tray (e.g. seals 30 or container bodies 32) may be arranged to align with the contents of another, corresponding tray. For example, the nested format of the one or more trays may be similar to a grid structure (e.g., a matrix with multiple rows and columns), such that each seal 30 or container body 32, for example, may be disposed in a corresponding portion or “square” of the grid. For example, the seal tray 26 may be a press-fit cap “nest” having a nested format (e.g., grid structure), where each cap (e.g., seal 30) may be disposed in a respective part of the grid. The container tray 34 may have a similar nested format, such that the grid of the seal tray 26 and the grid of the container tray 34 may be aligned so that each seal 30 may be concentrically paired with each corresponding container body 32. In this way, each tray (e.g. the container tray 34 and/or the seal tray 26) may support each of its respective contents (e.g. the container bodies 32 and/or the seals 30) in a manner that aligns the contents with its corresponding parts of another tray.

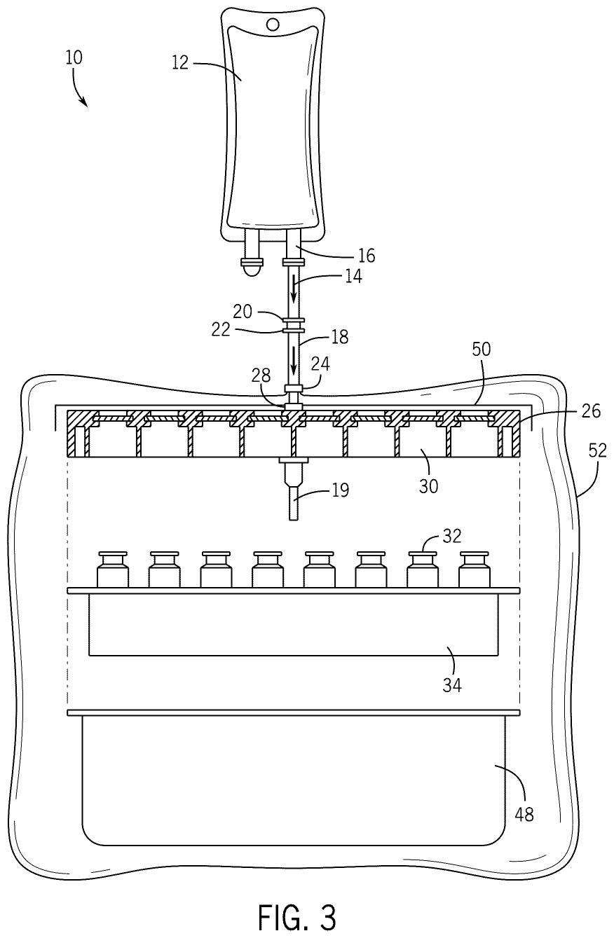

In some embodiments, one or more of the trays in the tray assembly 15 may not have a grid structure, but rather exhibit an open “tub” structure to serve as a housing for one or more other trays (e.g., a nested or grid tray). For example, as shown in FIG. 2, the container tray 34 may have a grid format, where each container body 32 is disposed in a respective portion of the container tray 34. The container tray 34 having the container bodies 32 may be disposed in a housing tray 48 (e.g., “tub”) configured to house the container tray 34. In such an embodiment, the housing tray 48 for the container tray 34 may serve as a bottom portion of the system 10, while the container tray 34 disposed inside the housing tray 48 may remain open such that the openings of each container body 32 is exposed. Conversely, in some embodiments, the seal tray 26 may be disposed in a housing tray similar to that of the housing tray 48 for the container tray 34. However, the housing tray for the seal tray 26 may serve as a top portion of the system 10, such that the bottom of the housing tray remains open so as to allow the seals 30 to make contact with the openings of the container bodies 32. FIG. 3 provides a visual of an embodiment where the seal tray 26 may be disposed in a housing tray 50 with an open bottom. Thus, all the trays in the tray assembly 15 (e.g., the seal tray 26, the container tray 34, and the one or more housing trays 48, 50) may be positioned inside the enclosure 52.

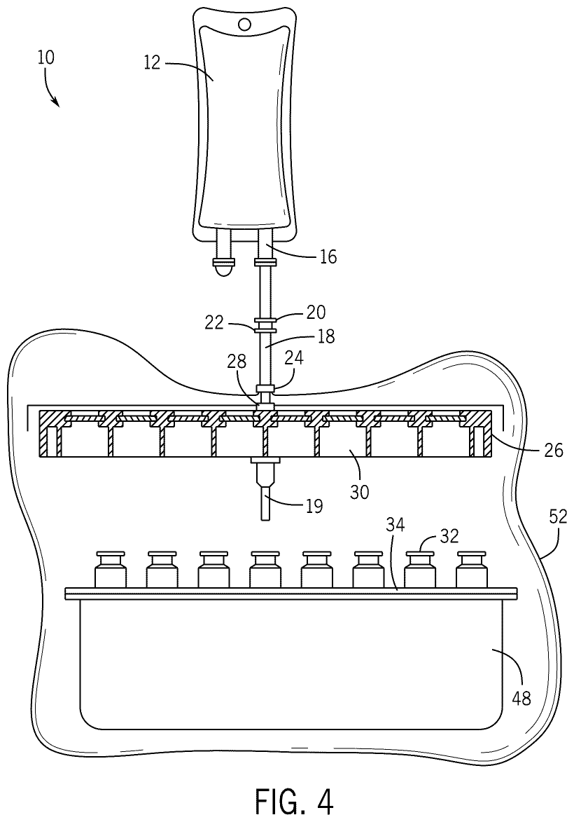

FIG. 4 is a diagram of a side view of the aseptic system of FIG. 1 during a dosing stage of manufacturing, according to an embodiment of the present disclosure. Once the system 10 is assembled and sterilized, an operator of the system 10 may begin the dosing process of manufacturing. For example, in the embodiment shown in FIG. 4, the fluid supply 12 may be fluidly coupled to the dosing conduit via one or more connectors 20, 22 connecting the fluid conduit 16 to the dosing conduit 18. The dosing conduit 18 and the dosing needle 19 may extend through the enclosure 52 and through the seal tray 26 (e.g. and a housing tray 50 housing the seal tray 26). A position of the dosing conduit 18 in the seal tray 26 may be fixed in place by one or more stoppers 28, such that the dosing conduit 18 and the dosing needle 19 are physically coupled to the seal tray 26. In embodiments where the enclosure 52 is flexible, or at least partially flexible, an operator and/or a machine (e.g., robotic arm assembly) may maneuver the dosing conduit 18 and/or dosing needle 19 and/or seal tray 26 to position the dosing needle 19 over an opening of a container body 32. The fluid 14 may be released from the fluid supply 12 and dispensed via the dosing needle 19 into a chamber of the container body 32. Thus, in this way, the flexible enclosure 52 may enable the movement of the seal tray 26 with the dosing conduit 18 relative to the container tray 34 to dispense a pharmaceutical product (e.g., the fluid 14) into the chamber of each of the plurality of container bodies 32, while the enclosure 52 contains and protects the dosing process from any external contamination. This dosing process of moving the seal tray 26, relative to the container tray 34, with the dosing conduit 18 and the dosing needle 19 via the enclosure 52 and dispense the fluid 14 into the chamber of the container bodies 32 may be repeated for each container body 32 in the container tray 34.

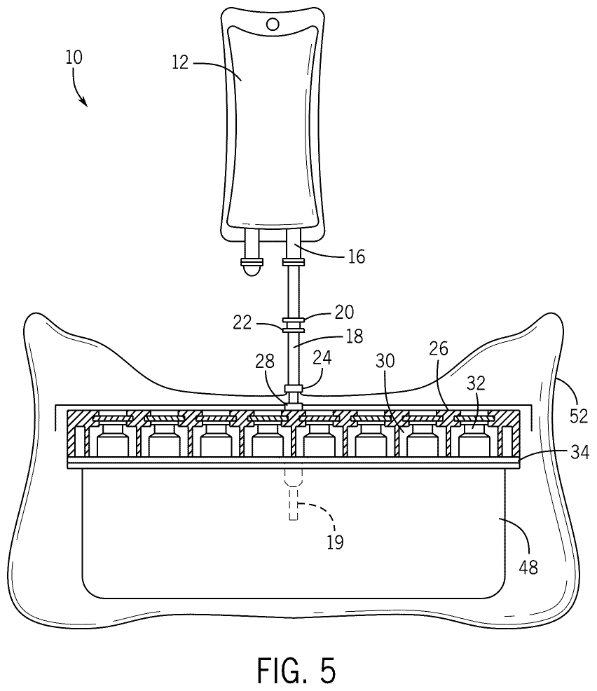

FIG. 5 is a diagram of the aseptic system of FIG. 1 during a sealing stage of manufacturing, according to an embodiment of the present disclosure. Once the dosing stage is complete and the container bodies 32 are filled with the fluid 14, the container bodies 32 may be sealed by the one or more seals 30 to form fluid-filled container assemblies 54 (e.g., fluid 14 contained by container bodies 32 and seals 30). For example, the operator and/or a machine (e.g., robotic arm assembly) may align the seals 30 with the openings of each container body 32 and couple the seal tray 26 with the container tray 34 in an axial direction (e.g. in a vertical direction). Once coupled, the operator and/or machine may seal each container body 32 by applying pressure or force on the seal tray 26. The enclosure 52 may be removed and disposed and the sealed, fluid-filled container bodies 54 may be removed from the container tray 34. Again, the enclosure 52 contains and protects the trays 26 and 34, the container bodies 32, the seals 30, and the fluid 14 from any external contamination, such that manufacturing of the fluid filled container assemblies (e.g., fluid 14 contained by container bodies 32 and seals 30) can be achieved in an aseptic environment without any specialized aseptic equipment.

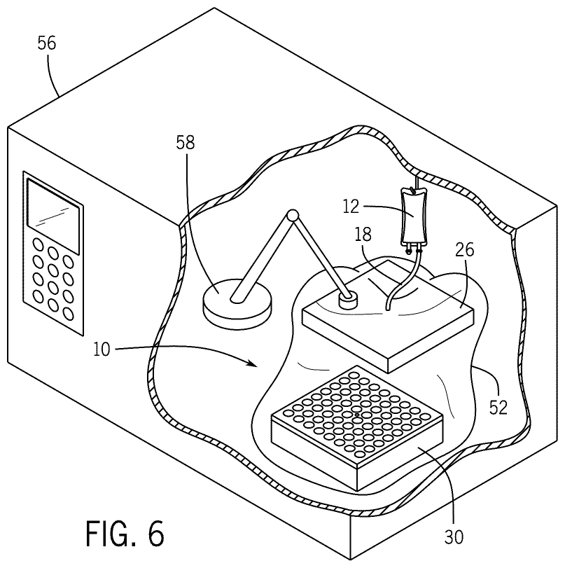

FIG. 6 illustrates a non-limiting embodiment of the aseptic system 10 of FIG. 1 for use in automated manufacturing of sterile injectables. As described above with regards to manually dosing and sealing the container bodies 32, the same process and procedures may be performed by automated machinery without requiring an aseptic chamber in the machinery. For example, as shown in FIG. 6, the system 10 may be implemented in an automated dosing system 56 equipped with automated machinery 58 (e.g., robotic arm) that is able to be programed or controlled to maneuver the seal tray 26 or the dosing needle 19 and dispense the fluid 14 into the chamber of each container body 32 using the enclosure 52 to maintain the aseptic environment, rather than requiring an aseptic environment with the automated dosing system 56. Further, the automated machinery 58 may be able to be programed or controlled to seal the container bodies 34 by aligning the seals 30 with the openings of the container bodies 32 and coupling the seal tray 26 with the container tray 34 in a manner similar to that described above.

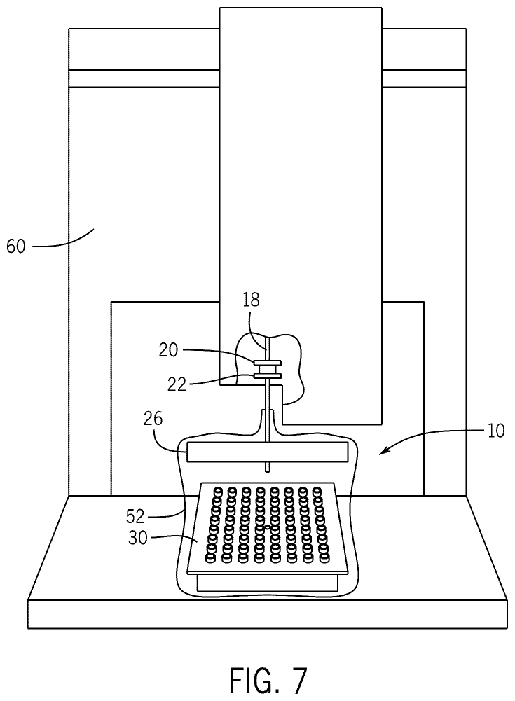

FIG. 7 illustrates another non-limiting embodiment of the system 10 for use in automated manufacturing of sterile injectables. For example, the system 10 may be implemented in a robotic liquid handling system 60 (e.g., an automated liquid handling platform) configured for dispensing fluid pharmaceuticals into one or more container bodies 32. The liquid handling system 60 may include or exclude a non-sterile, non-aseptic environment, because the enclosure 52 independently provides an aseptic environment as discussed above. For example, the enclosure 52 maintains an aseptic environment around the dosing system 17 and tray assembly 15. The liquid handling system 60 may be configured to automate a fluid dispensing protocol, thereby enabling the liquid handling system 60 to maneuver the seal tray 26, the dosing needle 19, and/or the dosing conduit 18 relative to the container tray 34 to position the dosing needle 19 over the opening of each container body 32 and dispense the fluid 14 into the container body 32 while the enclosure 52 maintains the aseptic environment. In some embodiments, the liquid handling system 60 may be configured to maneuver the container tray 34 to position the opening of each container body, one-by-one, under the dosing needle 19. In this way, the process of dosing and sealing the container bodies 32 (e.g. sterile injectables) may be manual or may be fully automated.

Technical effects of the disclosed embodiments include a sterilized, ready-to-use aseptic manufacturing system for dosing and packaging of a pharmaceutical product (e.g., medicine) in a container (e.g., vial). The disclosed embodiments enable manual and/or automated dosing of the pharmaceutical product without requiring a separate aseptic environment and/or aseptic dosing equipment. In particular, the sterilized, ready-to-use aseptic manufacturing system includes an enclosure (e.g., flexible bag) that houses or contains a seal tray (e.g., supporting seals or plugs), a container tray (e.g., tray supporting containers or vials), and at least a portion of a dosing system configured to dose the containers with the pharmaceutical product. The enclosure maintains an aseptic environment for the dosing process until the seals are coupled to and seal the containers, after which time the enclosure may be removed from the system. The sterilized, ready-to-use aseptic manufacturing system enables either manual dosing without any machinery and/or partially or entirely automated dosing with a machine (e.g., robotic arm assembly) without requiring an aseptic environment as part of the machine. Thus, the sterilized, ready-to-use aseptic manufacturing system disclosed herein reduces costs and complexity associated with the manufacturing of a pharmaceutical product. The sterilized, ready-to-use aseptic manufacturing system is particularly advantageous for small batches of pharmaceutical products at a healthcare center or other facility, such as small batches of 5, 10, 25, 50, 75, 100, 125, 150, 175, 200, 225, 250, 275, 300, 325, 350, 375, 400, 425, 450, 475, or 500 containers of the pharmaceutical product. However, the sterilized, ready-to-use aseptic manufacturing system may be used for any batch size, in any facility, using manual or automated dosing, or any combination thereof.

The subject matter described in detail above may be defined by one or more clauses, as set forth below.

An aseptic system includes a flexible enclosure and a tray assembly configured to be disposed within the flexible enclosure. The tray assembly includes a first tray configured to support a plurality of container bodies, wherein each of the plurality of container bodies has an opening into a chamber. The tray assembly further includes a second tray configured to support a plurality of container seals, wherein each of the plurality of container seals is configured to seal the opening of a respective one of the plurality of container bodies. The system further includes a dosing system having a dosing conduit extending through the flexible enclosure and the second tray. The flexible enclosure is configured to enable movement of the second tray with the dosing conduit relative to the first tray to dispense a pharmaceutical product into the chamber of each of the plurality of container bodies.

The system of the preceding clause, including the plurality of container bodies disposed in the first tray and the plurality of container seals disposed in the second tray.

The system of any preceding clause, wherein the plurality of container bodies includes a plurality of vials, syringes, bottles, bags, ampules, cartridges, or any combination thereof.

The system of any preceding clause, wherein the first tray includes a first grid portion disposed in a first housing portion and the second tray includes a second grid portion disposed in a second housing portion, wherein the first grid portion is configured to support the plurality of container bodies and the second grid portion is configured to support the plurality of container seals.

The system of any preceding clause, wherein the first housing portion is a bottom portion having an open top, and the second housing portion is a cover portion having an open bottom.

The system of any preceding clause, wherein the flexible enclosure includes a transparent or translucent material.

The system of any preceding clause, wherein the flexible enclosure includes a flexible bag.

The system of any preceding clause, wherein the flexible enclosure includes a flexible wall that defines at least 50 percent of the flexible enclosure.

The system of any preceding clause, wherein the flexible enclosure includes a flexible wall that defines at least 90 percent of the flexible enclosure.

The system of any preceding clause, wherein the flexible enclosure has a flexible wall portion and a rigid wall portion.

The system of any preceding clause, wherein the flexible enclosure is disposable.

The system of any preceding clause, wherein the first tray and the second tray are configured to align the plurality of container bodies with the respective plurality of container seals.

The system of any preceding clause, including the pharmaceutical product disposed in each of the container bodies via dosing by the dosing system.

The system of any preceding clause, wherein the first and second trays are coupled together in an axial direction relative to axes of the plurality of container bodies, and the plurality of container seals are configured to seal the openings in the respective plurality of container bodies in response to coupling together the first and second trays in the axial direction.

The system of any preceding clause, wherein the flexible enclosure is removable after plurality of container bodies are sealed by the plurality of container seals.

A method includes supporting a plurality of container bodies in a first tray of a tray assembly, wherein each of the plurality of container bodies has an opening into a chamber. The method further includes supporting a plurality of container seals in a second tray of the tray assembly, wherein each of the plurality of container seals is configured to seal the opening of a respective one of the plurality of container bodies. The method further includes positioning the tray assembly within a flexible enclosure, and extending a dosing conduit of a dosing system through the flexible enclosure and the second tray. The flexible enclosure is configured to enable movement of the second tray with the dosing conduit to dispense a pharmaceutical product into the chamber of each of the plurality of container bodies.

The method of the preceding clause, including sterilizing an assembly of the tray assembly, the flexible enclosure, and the dosing system.

The method of any preceding clause, including moving the second tray with the dosing conduit via the flexible enclosure to align the dosing conduit with one of the plurality of container bodies, dispensing the pharmaceutical product into the chamber of the one of the plurality of container bodies, and repeating the moving and dispensing for each of the plurality of container bodies.

An aseptic system, includes at least one component of a tray assembly configured to be disposed within a flexible enclosure. The system further includes a dosing system having a dosing conduit configured to extend through the flexible enclosure and the at least one component. The flexible enclosure is configured to enable movement of the at least one component with the dosing conduit to dispense a pharmaceutical product into a chamber of each of a plurality of container bodies disposed in the tray assembly.

The system of the preceding clause, wherein the tray assembly includes a first tray configured to support the plurality of container bodies, wherein each of the plurality of container bodies has an opening into the chamber. The system further includes second tray configured to support a plurality of container seals, wherein each of the plurality of container seals is configured to seal the opening of a respective one of the plurality of container bodies.

While the embodiments set forth in the present disclosure may be susceptible to various modifications and alternative forms, specific embodiments have been shown by way of example in the drawings and have been described in detail herein. However, it should be understood that the disclosure is not intended to be limited to the particular forms disclosed. The disclosure is to cover all modifications, equivalents, and alternatives falling within the spirit and scope of the disclosure as defined by the following appended claims.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S. C. § 112(f).

1. An aseptic system comprising:
a flexible enclosure;
a tray assembly configured to be disposed within the flexible enclosure, wherein the tray assembly comprises:
a first tray configured to support a plurality of container bodies, wherein each of the plurality of container bodies has an opening into a chamber; and
a second tray configured to support a plurality of container seals, wherein each of the plurality of container seals is configured to seal the opening of a respective one of the plurality of container bodies; and
a dosing system comprising a dosing conduit extending through the flexible enclosure and the second tray, wherein the flexible enclosure is configured to enable movement of the second tray with the dosing conduit relative to the first tray to dispense a pharmaceutical product into the chamber of each of the plurality of container bodies.

2. The system of claim 1, comprising the plurality of container bodies disposed in the first tray and the plurality of container seals disposed in the second tray.

3. The system of claim 2, wherein the plurality of container bodies comprises a plurality of vials, syringes, bottles, bags, ampules, cartridges, or any combination thereof.

4. The system of claim 1, wherein the first tray comprises a first grid portion disposed in a first housing portion and the second tray comprises a second grid portion disposed in a second housing portion, wherein the first grid portion is configured to support the plurality of container bodies and the second grid portion is configured to support the plurality of container seals.

5. The system of claim 4, wherein the first housing portion is a bottom portion having an open top, and the second housing portion is a cover portion having an open bottom.

6. The system of claim 1, wherein the flexible enclosure comprises a transparent or translucent material.

7. The system of claim 1, wherein the flexible enclosure comprises a flexible bag.

8. The system of claim 1, wherein the flexible enclosure comprises a flexible wall that defines at least 50 percent of the flexible enclosure.

9. The system of claim 1, wherein the flexible enclosure comprises a flexible wall that defines at least 90 percent of the flexible enclosure.

10. The system of claim 1, wherein the flexible enclosure has a flexible wall portion and a rigid wall portion.

11. The system of claim 1, wherein the flexible enclosure is disposable.

12. The system of claim 1, wherein the first tray and the second tray are configured to align the plurality of container bodies with the respective plurality of container seals.

13. The system of claim 2, comprising the pharmaceutical product disposed in each of the container bodies via dosing by the dosing system.

14. The system of claim 13, wherein the first and second trays are coupled together in an axial direction relative to axes of the plurality of container bodies, and the plurality of container seals are configured to seal the openings in the respective plurality of container bodies in response to coupling together the first and second trays in the axial direction.

15. The system of claim 14, wherein the flexible enclosure is removable after plurality of container bodies are sealed by the plurality of container seals.

16. A method, comprising:
supporting a plurality of container bodies in a first tray of a tray assembly, wherein each of the plurality of container bodies has an opening into a chamber; and
supporting a plurality of container seals in a second tray of the tray assembly, wherein each of the plurality of container seals is configured to seal the opening of a respective one of the plurality of container bodies;
positioning the tray assembly within a flexible enclosure; and
extending a dosing conduit of a dosing system through the flexible enclosure and the second tray, wherein the flexible enclosure is configured to enable movement of the second tray with the dosing conduit to dispense a pharmaceutical product into the chamber of each of the plurality of container bodies.

17. The method of claim 16, comprising sterilizing an assembly of the tray assembly, the flexible enclosure, and the dosing system.

18. The method of claim 16, comprising:
moving the second tray with the dosing conduit via the flexible enclosure to align the dosing conduit with one of the plurality of container bodies; and
dispensing the pharmaceutical product into the chamber of the one of the plurality of container bodies; and
repeating the moving and dispensing for each of the plurality of container bodies.

19. An aseptic system, comprising:
at least one component of a tray assembly configured to be disposed within a flexible enclosure; and
a dosing system comprising a dosing conduit configured to extend through the flexible enclosure and the at least one component, wherein the flexible enclosure is configured to enable movement of the at least one component with the dosing conduit to dispense a pharmaceutical product into a chamber of each of a plurality of container bodies disposed in the tray assembly.

20. The system of claim 19, wherein the tray assembly comprises:
a first tray configured to support the plurality of container bodies, wherein each of the plurality of container bodies has an opening into the chamber; and
a second tray configured to support a plurality of container seals, wherein each of the plurality of container seals is configured to seal the opening of a respective one of the plurality of container bodies.
